(19) United States
(12) Patent Application Publication
(10) Pub. No.: US 2010/0221732 A1
(43) Pub. Date: Sep. 2, 2010

(54) METHODS AND COMPOSITIONS FOR EVALUATING CHRONIC IMMUNE DISEASES

(76) Inventors: Marc Fremont, Zellik (BE); Kenny Leo De Meirleir, Mechelen (BE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/713,614

(22) Filed: Feb. 26, 2010

Related U.S. Application Data
(60) Provisional application No. 61/156,346, filed on Feb. 27, 2009.

(51) Int. Cl. C12Q 1/68; C12M 1/34

(52) U.S. Cl. 435/6; 435/287.2

(57) ABSTRACT

Methods and compositions are provided for evaluating a subject for chronic immune disease, including predicting whether a subject is susceptible to a chronic immune disease, diagnosing whether a subject has a chronic immune disease and/or determining a treatment for a subject suffering from a chronic immune disease. Aspects of the methods include genotyping a subject to determine whether the subject has a least one polymorphism in at least one gene chosen from TLR4, Hsp60, IL-23R, IL-6 and IL-17F. In addition, reagents, devices and kits thereof that find use in practicing the subject methods are provided.

### CROSS REFERENCE To RELATED APPLICATIONS

Pursuant to 35 U.S.C. §119 (e), this application claims priority to the filing date of the U.S. Provisional Patent Application Ser. No. 61/156,346 filed Feb. 27, 2009; the disclosure of which are herein incorporated by reference.

### INTRODUCTION

Chronic Fatigue Syndrome (CFS)/myalgic encephalomyelitis is defined by a severe and debilitating fatigue associated with a variety of symptoms including musculoskeletal pain, sore throat, tender lymph nodes, sleep abnormalities, and neurocognitive problems (Fukuda et al. (1994) Ann Intern Med 121:953-959). The pathogenesis of CFS is still poorly understood, but is likely to be multifactorial; viral infections, stress, neuroendocrine dysfunctions, exposure to toxins have all been proposed as contributing factors to the onset and maintenance of the disease (Devanur and Kerr (2006) J Clin Virol 37(3):139-50).

### SUMMARY

Methods and compositions are provided for evaluating a subject for a chronic immune disease, including predicting whether a subject is susceptible to a chronic immune disease, diagnosing whether a subject has a chronic immune disease and/or determining a treatment for a subject suffering from a chronic immune disease. Aspects of the methods include genotyping a subject to determine whether the subject has a least one polymorphism in at least one gene chosen from TLR4, Hsp60, IL-23R, IL-6 and IL-17F. In addition, reagents, devices and kits thereof that find use in practicing the subject methods are provided.

### DETAILED DESCRIPTION

Methods and compositions are provided for evaluating a subject for a chronic immune disease, including predicting whether a subject is susceptible to a chronic immune disease, diagnosing whether a subject has a chronic immune disease and/or determining a treatment regimen for a subject suffering from a chronic immune disease. Aspects of the methods include genotyping a subject to determine whether the subject has a least one polymorphism in at least one gene chosen from TLR4, Hsp60, IL-23R, IL-6 and IL-17F. In addition, reagents, devices and kits thereof that find use in practicing the subject methods are provided.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the subject invention is directed to methods of evaluating chronic immune disease including predicting whether a subject is susceptible to a chronic immune disease, diagnosing whether a subject has a chronic immune disease or determining a treatment for a subject suffering from a chronic immune disease, as well as reagents and kits thereof (and devices) for use in practicing the subject methods. In further describing the invention, the subject methods are described first, followed by a review of the reagents, kits and devices for use in practicing the subject methods.

### Methods of Evaluating a Subject for Chronic Immune Disease

Embodiments of the subject invention are directed to methods for: (a) predicting whether a subject is susceptible to a chronic immune disease; (b) diagnosing a subject as having a chronic immune disease; and (c) determining a treatment in a subject suffering from a chronic immune disease. Examples of chronic immune diseases are Chronic Fatigue Syndrome and Multiple Sclerosis. In further describing aspects of the invention, the following description focuses on Chronic Fatigue Syndrome. However, the subject methods and the reagents, devices and kits thereof also find use in the prediction of susceptibility to, diagnosis of, and treatment of other chronic immune diseases as well, as described above.

Chronic Fatigue Syndrome (CFS), also known as myalgic encephalomyelitis (ME), is a condition in which the subject suffers from fatigue associated with symptoms which may include but are not limited to muscle and joint (musculoskeletal) pain, sore throat, tender lymph nodes, sleep abnormalities, and/or neurocognitive problems. Often, the symptoms last for six months or more. Often, the fatigue is debilitating. In certain embodiments, the methods are directed at CFS and particularly at whether a subject will or will not be susceptible to CFS, at diagnosing a subject as having CFS, or at determining what treatment to provide a subject with CFS.

Aspects of the invention include methods of predicting whether a subject is or is not susceptible to CFS, or is or is not resistant to CFS. A subject is susceptible to CFS if the subject is more likely to develop CFS than an individual of the general population, whereas a subject that is resistant to CFS is less likely to develop CFS than an individual of the general population.

Additional aspects of the invention include methods of diagnosing whether a subject suspected of having CFS has CFS. Subjects suspected of having CFS and thus amenable to these methods can be identified using any convenient protocol. One convenient protocol is diagnosis based on clinical symptoms. A number of different clinical symptoms may be used to identify subjects that may have or have CFS. For example, clinical symptoms of interest include: fatigue of six months or longer that causes a reduction in effort of greater than 50 percent of normal output, pain in multiple joints (arthralgia), muscle pain (myalgia), frequent or recurring sore throat, or tender cervical or axillary lymph nodes, headaches of a new kind or greater severity, cognitive dysfunction (e.g. memory loss, impaired concentration); and the like. The presence of one or more of the above symptoms may be used to identify subjects suspected of having CFS.

Aspects of the invention further include methods of determining a treatment protocol/regimen for a subject suffering from CFS. The subject methods may be used to predict the risk of a CFS complication, where that result may be used to determine a treatment protocol. In other words, the subject methods may be used to predict the risk of a CFS subject developing a complication, the results of such a prediction being useful in determining a prophylactic treatment to prevent the complication. The subject methods may also be used to diagnose the cause of an existing complication, wherein that result may be used to determine a treatment for that complication. A complication is an unfavorable evolution of a disease, a health condition or a medical treatment. Complications could be a natural evolution of CFS, or a result of a medical treatment that produces adverse effects and/or produces new health problem(s) by itself, or a new disease that develops as a result of CFS. Indications of complications include but are not limited to indications that the CFS has become worse in its severity or shows a increased number of signs, symptoms or new pathologies.

In practicing methods according to certain embodiments, a subject or patient sample, e.g., cells or collections thereof, e.g., tissues, is assayed to determine whether the host or subject from which the assayed sample was obtained is susceptible to CFS, or to diagnose a subject as having CFS, or determine a treatment for a person suffering from CFS. In practicing the subject diagnostic methods, the sample obtained from the host is assayed to determine the genotype of the host or subject from which the sample was obtained with respect to at least one, i.e., one or more, polymorphisms, where polymorphisms of interest are referred to herein as target polymorphisms. In certain embodiments, the at least one target polymorphism is in the TLR4, Hsp60, IL-23R, IL-6 or IL-17F gene.

A TLR4 polymorphism is a polymorphism present in the TLR4 (Toll-like receptor 4) protein. The amino acid sequence for TLR4 and the nucleic acid sequence that encodes it are deposited at Genbank NM_{—}138554 (SEQ ID NO:1 and SEQ ID NO:2). TLR4 is a type I transmembrane protein that acts as a cell surface receptor, for example, as a pattern recognition receptor (PRR) on immune cells for pathogen associated molecular patterns (PAMPs), wherein recognition and binding of PAMPs by TLR4 often induces a pro-inflammatory cellular response. Often, recognition of PAMPs occurs when TLR4 heterodimerizes with another TLR. Polymorphisms of interest include but are not limited to those that modulate TLR4 function such that cellular responsiveness to, e.g. LPS, is diminished, including those that modulate TLR4 recognition of PAMPs, those that modulate dimerization of TLR4 with other TLRs, and those that modulate downstream signaling mechanism by which TLR4 stimulates a cell, for example, the rs4986790 polymorphism (Asp299Gly), wherein a Gly at residue 299 is correlated with a diminished cellular response to LPS and is predictive of susceptibility to CFS. Polymorphisms of particular interest include the rs4986790 A/G polymorphism, in which the A allele encodes a variant with an Asp at residue 299 and the G allele encodes a variant with a Gly at residue 299 (“Asp299Gly”), wherein the G allele is predictive of susceptibility to CFS; and the rs12344353 T/C polymorphism, in which there is a T/C genetic polymorphism in the first intron (i.e. the intron between exons 1 and 2) of the TL4R gene, where the C allele is predictive of susceptibility to CFS.

An Hsp60 polymorphism is a polymorphism present in the Hsp60 (Heat shock protein 60) protein. The amino acid sequence for Hsp60, also known as HSPD1 and chaperonin 60-kD (CPN60), and variants, and the nucleic acid sequences that encode them are deposited at Genbank NM_{—}002156 (SEQ ID NO:3 and SEQ ID NO:4) and NM_{—}199440 (SEQ ID NO:5 and SEQ ID NO:6). Hsp60 is a molecular chaperone, acting, for example, to assist in protein folding and in replication and transmission of mitochondrial DNA. Polymorphisms of interest include but are not limited to those that modulate Hsp60 function, for example, by modulating binding with proteins they are chaperoning, by modulating binding with ATP, by modulating binding with chaperonin proteins, or by modulating binding with single strand DNA. Polymorphisms of particular interest include the rs2340690 T/C polymorphism, in which there is a T/C genetic polymorphism in an intron of the Hsp60 gene, where two T alleles is predictive of susceptibility to CFS.

An IL-23R polymorphism is a polymorphism present in the IL-23R (interleukin 23 receptor) protein. The amino acid sequence for IL-23R and the nucleic acid sequence that encodes it are deposited at Genbank NM_{—}144701 (SEQ ID NO:7 and SEQ ID NO:8). IL-23R is a type 1 transmembrane protein that acts as a receptor for cytokines including IL-23, wherein receptor ligand binding by IL-23R often induces a pro-inflammatory response. Polymorphisms of interest include but are not limited to those that modulate IL-23R function, for example, by modulating IL-23R binding to its ligands or by modulating downstream signaling. Polymorphisms of particular interest include the rs11209026 G/A polymorphism, wherein the G allele encodes a variant with an Arg at residue 381 and the A allele encodes a variant with a Gln at residue 381 (“Arg381 Gln”, or “R381Q”), where the A allele is predictive of susceptibility to CFS.

An IL-6 polymorphism is a polymorphism present in the IL-6 (interleukin 6) protein. The amino acid amino acid sequence for IL-6, also known as interferon beta-2 (IFNβ2), B-cell stimulatory factor (BSF2), hepatocyte stimulatory factor (HSF), and hybridoma growth factor (HGF), and the nucleic acid sequence that encodes it are deposited at Genbank NM_{—}000600 (SEQ ID NO:9 and SEQ ID NO:10). IL-6 is a cytokine that often produces a proinflammatory cellular response. Polymorphisms of interest include but are not limited to those which modulate IL-6 function, for example, by increasing IL-6 binding to its receptor or increasing IL-6 levels in the blood, as with, for example, the rs1800795 polymorphism, in which the G allele enhances promoter activity, resulting in increased levels of IL-6 in serum. Polymorphisms of particular interest include the rs1800795 C/G polymorphism, in which there is a C/G genetic polymorphism in the promoter region of the IL-6 gene, where the G allele is predictive of susceptibility to CFS.

An IL-17F polymorphism is a polymorphism present in the IL-17F (Interleukin 17F) protein. The amino acid sequence for IL-17F the nucleic acid sequence that encodes it are deposited at Genbank NM_{—}052872 (SEQ ID NO:11 and SEQ ID NO:12). IL-17F is a cytokine that often produces a proinflammatory response. Polymorphisms of interest include but are not limited to polymorphisms that encode amino acid substitutions that modulate IL-17F function, for example, by antagonizing IL-17F function, e.g. proinflammatory effects, e.g. by diminishing IL-17F binding to its receptor or reducing IL-17F levels in the blood such that cellular response is diminished, as with, for example, the rs763780 polymorphism, in which an Arg at residue 161 is associated with diminished cellular response. Polymorphisms of particular interest include the rs763780 T/C polymorphism, wherein the T allele encodes a variant with a His at residue 161 and the C allele encodes a variant with an Arg at residue 161 (“His161Arg” or “H161R”), where the C allele confers resistance to CFS.

More information about the aforementioned polymorphisms as well as other polymorphisms in the aforementioned genes that may be of interest in the present invention may be found in the NCBI SNP database, searchable at the website having an address produced by placing “http://www.” in front of “ncbi.nlm.nih.gov/PubMed/”, using the polymorphism or the gene name as the search term and clicking on the link to the “gene view” in any entry.

In certain embodiments, the sample is assayed to determine the genotype of the host with respect to a single target polymorphism. In these embodiments, the single target polymorphism is a polymorphism in the TLR4, Hsp60, IL-23R, IL-6 or IL-17F gene. In certain embodiments, the sample is assayed to determine the genotype of the host with respect to two or more different target polymorphisms, where in these embodiments, the two or more different target polymorphisms are polymorphisms in the TLR4, Hsp60, IL-23R, IL-6 or IL-17F genes. In certain of these embodiments, at least two of the target polymorphisms are polymorphisms in different genes. In certain embodiments, the sample is assayed for both a first and a second polymorphism

Any convenient protocol for assaying a sample for the above one or more target polymorphisms may be employed in the subject methods. In certain embodiments, the target polymorphism is detected at the protein level, e.g., by assaying for a polymorphic protein. In yet other embodiments, the target polymorphism is detected at the nucleic acid level, e.g., by assaying for the presence of nucleic acid polymorphism, e.g., a single nucleotide polymorphism (SNP) that causes expression of the polymorphic protein.

For example, polynucleotide samples derived from (e.g., obtained from) an individual may be employed. Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein. Detection of a target polymorphism in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; and/or an alteration in corresponding polypeptide activity.

Detection of a target polymorphism by analyzing a polynucleotide sample can be conducted in a number of ways. A test nucleic acid sample can be amplified with primers which amplify a region known to comprise the target polymorphism(s). Genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Once the region comprising a target polymorphism has been amplified, the target polymorphism can be detected in the PCR product by nucleotide sequencing, by Single Strand Conformation Polymorphism (SSCP) analysis, or any other method known in the art. In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease. PCR may also be used to determine whether a polymorphism is present by using a primer that is specific for the polymorphism. Such methods may comprise the steps of collecting from an individual a biological sample comprising the individual's genetic material as template, optionally isolating template nucleic acid (genomic DNA, mRNA, or both) from the biological sample, contacting the template nucleic acid sample with one or more primers that specifically hybridize with a target polymorphic nucleic acid molecule under conditions such that hybridization and amplification of the template nucleic acid molecules in the sample occurs, and detecting the presence, absence, and/or relative amount of an amplification product and comparing the length to a control sample. Observation of an amplification product of the expected size is an indication that the target polymorphism contained within the target polymorphic primer is present in the test nucleic acid sample. Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230. As one non-limiting example, a PCR primer comprising the T78C variation described in Example 1 may be used.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid may be sequenced by a dideoxy chain termination method or other well-known methods. Genomic DNA or mRNA may be used directly. If mRNA is used, a cDNA copy may first be made. If desired, the sample nucleic acid can be amplified using a PCR. A variety of sequencing reactions known in the art can be used to directly sequence the relevant gene, or a portion thereof in which a specific polymorphism is known to occur, and detect polymorphisms by comparing the sequence of the sample nucleic acid with a reference polynucleotide that contains a target polymorphism. Any of a variety of automated sequencing procedures can be used. See, e.g., WO 94/16101; Cohen et al. (1996) Adv. Chromatography 36:127-162.

Hybridization with the variant sequence may also be used to determine the presence of a target polymorphism. Hybridization analysis can be carried out in a number of different ways including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences. Identification of a polymorphism in a nucleic acid sample can be performed by hybridizing a sample and control nucleic acids to high density arrays containing hundreds or thousands of oligonucleotide probes. Cronin et al. (1996) Human Mutation 7:244-255; and Kozal et al. (1996) Nature Med. 2:753-759.

Single strand conformational polymorphism (SSCP) analysis; denaturing gradient gel electrophoresis (DGGE); mismatch cleavage detection; and heteroduplex analysis in gel matrices can also be used to detect polymorphisms. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels. The aforementioned techniques are well known in the art. Detailed description of these techniques can be found in a variety of publications, including, e.g., “Laboratory Methods for the Detection of Mutations and Polymorphisms in DNA” (1997) G. R. Taylor, ed., CRC Press, and references cited therein.

A number of methods are available for determining the expression level of a polymorphic nucleic acid molecule, e.g., a polymorphic polypeptide in a particular sample. Diagnosis may be performed by a number of methods to determine the absence or presence or altered amounts of normal or abnormal mRNA in a patient sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc. Detailed descriptions of how to make antibodies can be found in a variety of publications, including, e.g. “Making and using Antibodies: A Practical Handbook” (2006) G. C. Howard and M. R. Kaser, eds. CRC Press; “Antibody Engineering: Methods and Protocols” (2004) B. K. C. Lo, ed, Humana Press.

Alternatively, one may focus on the expression of mRNA. Biochemical studies may be performed to determine whether a sequence polymorphism in a coding region or control regions of interest is associated with disease. Disease associated polymorphisms may include deletion or truncation of the gene, mutations that alter expression level, that affect the activity of the protein, etc.

Alternatively, one may screen for polymorphisms at the protein level. Screening for mutations in a polymorphic polypeptide may be based on the functional or antigenic characteristics of the protein. Functional assays include cofactor binding assays, enzyme activity assays, substrate binding assays or surface expression assays. For example, protein truncation assays are useful in detecting deletions that may affect the biological activity of the protein. The activity of the encoded a polymorphic polypeptide may be determined by comparison with a reference polypeptide lacking a specific polymorphism. Alternatively, the three-dimensional structure of the protein may be assayed, for example by fluorescence polarization or circular dichroism spectroscopy, wherein the three-dimensional structure of the encoded a polymorphic polypeptide may be determined by comparison with purified protein carrying the opposing allele of the polymorphism. Alternatively, various immunoassays designed to detect polymorphisms in polymorphic polypeptides may be used. The absence or presence of antibody binding to a polymorphic polypeptide may be determined by various methods, including flow cytometry of dissociated cells, microscopy, radiography, scintillation counting, etc. Detailed descriptions of how to make antibodies, including antibodies that are specific for epitopes, for example, single amino acid substitutions within epitopes, can be found in a variety of publications, including, e.g. “Making and using Antibodies: A Practical Handbook” (2006) G. C. Howard and M. R. Kaser, eds. CRC Press; “Antibody Engineering: Methods and Protocols” (2004) B. K. C. Lo, ed, Humana Press; and U.S. Pat. No. 6,054,632, the disclosure of which is herein incorporated by reference.

Diagnostic methods of the subject invention in which the level of polymorphic gene expression is of interest will typically involve comparison of the relevant nucleic acid abundance of a sample of interest with that of a control value to determine any relative differences, where the difference may be measured qualitatively and/or quantitatively, which differences are then related to the presence or absence of an abnormal gene expression pattern. A variety of different methods for determining the nucleic acid abundance in a sample are known to those of skill in the art, where particular methods of interest include those described in: Pietu et al., Genome Res. (June 1996) 6: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 19960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

Additional references describing various protocols for detecting the presence of a target polymorphism include, but are not limited to, those described in: U.S. Pat. Nos. 6,703,228; 6,692,909; 6,670,464; 6,660,476; 6,653,079; 6,632,606; 6,573,049; the disclosures of which are herein incorporated by reference.

Following obtainment of the genotype from the sample being assayed, the genotype is evaluated to determine if the subject is susceptible/resistant to CFS, or to determine a treatment in a subject that is suffering from CFS. In certain embodiments, the obtained genotype may be compared with a reference or control to make a diagnosis regarding the CFS phenotype of the cell or tissue, and therefore host, from which the sample was obtained/derived. The terms “reference” and “control” as used herein mean a standardized genotype to be used to interpret the genotype of a given patient and assign a prognostic class thereto. The reference or control may be a genotype that is obtained from a cell/tissue known to have a particular phenotype, e.g., a susceptibility phenotype, and therefore may be a positive reference or control genotype. Alternatively, the reference/control genotype may be from a cell/tissue known to not have the desired phenotype, and therefore be a negative reference/control genotype.

In certain embodiments, the obtained genotype is compared to a single reference/control genotype to obtain information regarding the phenotype of the cell/tissue being assayed. In yet other embodiments, the obtained genotype is compared to two or more different reference/control profiles to obtain more in depth information regarding the phenotype of the assayed cell/tissue. For example, the obtained genotype may be compared to a positive genotype and a negative genotype to obtain confirmed information regarding whether the cell/tissue has the phenotype of interest.

Representative examples of genotypes associated with susceptibility to or with having CFS include, but are not limited to: the G allele of TLR4 polymorphism rs4986790, the C allele of TLR4 polymorphism rs12344353, two T alleles of Hsp60 polymorphism rs2340690, the A allele of IL-23R polymorphism rs11209026, and two G alleles of the IL-6 polymorphism rs1800795. Representative examples of genotypes associated with resistance to CFS include, but are not limited to the C allele of the IL-17F polymorphism rs763780.

In certain embodiments, the above-obtained information about the cell/tissue being assayed is employed to determine if the subject is susceptible/resistant to CFS, or to diagnose a subject with CFS, or to predict if a subject suffering from CFS will develop a particular complication. In certain embodiments, the above-obtained information is employed to give a refined probability determination as to whether a subject is susceptible to CFS, or to diagnose a subject with CFS, or to give a refined determination as to whether a subject suffering from CFS will develop a particular complication. For example, an identification of at least one G allele of TLR4 polymorphism rs4986790, at least one C allele of TLR4 polymorphism rs12344353, two T alleles of Hsp60 polymorphism rs2340690, at least one A allele of IL-23R polymorphism rs11209026, or two G alleles of the IL-6 polymorphism rs1800795 may be employed to determine that the subject has at least a 70% chance, such as at least a 75% chance, including at least an 80% chance of developing CFS or a complication from CFS. Likewise, an identification of at least one C allele of the IL-17F polymorphism rs763780 may be employed to determine that the subject has less than 50% chance, such as a less than 45% chance, including a less than 40% chance of developing CFS or a complication from CFS.

In some embodiments, determining that the subject is susceptible/resistant to CFS, or diagnosing a subject with CFS, or predicting if a subject suffering from CFS will develop a particular complication may be made by employing the results of the aforementioned genotype analysis in conjunction with the identification of symptoms known in the art to be associated with CFS, e.g. a new onset (not lifelong) of unexplained, persistent fatigue unrelated to exertion and not substantially relieved by rest, impaired memory or concentration, post-exertional malaise, where physical or mental exertions bring on “extreme, prolonged exhaustion and sickness”, unrefreshing sleep, muscle pain (myalgia), pain in multiple joints (arthralgia), headaches of a new kind or greater severity, frequent or recurring sore throat, or tender cervical or axillary lymph nodes. In addition, the artisan may consider the presence of symptoms that are attributable to other conditions, so as to exclude a determination of susceptibility to CFS or a CFS diagnosis, e.g. symptoms of mononucleosis, Lyme disease, lupus, multiple sclerosis, fibromyalgia, primary sleep disorders, severe obesity and major depressive disorders, as they are known in the art.

In some embodiments, the above-obtained information about the cell/tissue being assayed is employed to determine a treatment for a subject suffering from CFS. Treatments of interest include, but are not limited to, antihistamines, prescription sleep medications, analgesics, anti-depressants, dietary supplements, alternative medicine, cognitive behavior therapy (a form of psychological therapy used to treat chronically ill subjects), graded exercise therapy (a form of physical therapy), and pacing (an energy management strategy in which subjects are advised to set manageable daily activity/exercise goals and balance activity and rest to avoid over-exertion which may worsen symptoms). Classes of therapeutic treatments of interest include, but are not limited to, therapeutic antibodies, peptide mimetics, small molecule agonists or antagonists, siRNA-based therapeutics, and gene therapy.

In some instances, the treatment may be a pharmacogenomic treatment. In these applications, a subject/host/patient is first diagnosed for the presence of absence of a CFS phenotype using a protocol such as the diagnostic protocol described in the preceding section. The subject is then treated using a pharmacological protocol, where the suitability of the protocol for a particular subject/patient is determined using the results of the diagnosis step. In other words, the therapeutic protocol employed is a protocol that is tailored to the particular polymorphism identified in the patient sample. More specifically, where the identified phenotype is a polymorphism in one gene, a particular therapeutic treatment protocol is then employed to treat the subject. Alternatively, where a subject is identified as having a polymorphism in another gene, a different therapeutic treatment protocol is then employed. For example, a therapy for CFS or a CFS complication in a subject with a TLR4 polymorphism may include augmenting TLR4 signaling, for example with peptide mimetic or antibody therapy to promote signaling via the TLR4 Gly variant. As another example, a therapy for CFS or a CFS complication in a subject with an IL-6 rs18000795 polymorphism may include attenuating IL-6 signaling, for example with an IL-6 or IL-6 receptor blocking antibody, an IL-6-specific or IL-6 receptor-specific siRNA, or an IL-6 or IL-6 dominant negative peptide.

In some embodiments, providing an evaluation of a subject for CFS, e.g. a prediction of a subject's susceptibility to developing CFS, a diagnosis of CFS in a subject suspected of having CFS, and/or a determination of a treatment regimen for a CFS subject, includes generating a written report that includes the artisan's assessment of the subject's susceptibility to developing CFS, i.e. a “susceptibility assessment”, the subject's current state of health i.e. a “CFS diagnosis assessment”, and/or possible treatment regimens, i.e. a “treatment assessment”. Thus, a subject method may further include a step of generating or outputting a report providing the results of a susceptibility assessment, a CFS diagnosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a susceptibility assessment, a CFS diagnosis assessment, or treatment assessment and its results. A subject report can be completely or partially electronically generated. A subject report includes at least a susceptibility prediction, i.e. a prediction as to the susceptibility of a subject to developing CFS; or a CFS diagnosis, i.e. whether a subject does or does not have CFS; or a suggested treatment regimen, i.e. a course of treatment to be followed. A subject report can further include one or more of: 1) information regarding the testing facility; 2) service provider information; 3) subject data; 4) sample data; 5) an assessment report, which can include various information including: a) test data, where test data can include genotypes for one or more polymorphism in at least one gene chosen from TLR4, Hsp60, IL-23R, IL-6 and IL-17F and b) reference values employed, if any; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; or a tissue sample, e.g. a tissue biopsy, a swab of the lining of the mouth or nose, a collect hair follicle, etc. from a subject. Data generation can include genotyping for one or more polymorphisms in TLR4, Hsp60, IL-23R, IL-6 and/or IL-17F. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the susceptibility prediction, CFS diagnosis, or proposed treatment regimen) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed in the susceptibility prediction, such as the source of biological sample obtained from the subject (e.g. blood, saliva, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prediction of the likelihood that the patient will develop CFS. The interpretive report can include, for example, results of genotyping for a TLR4 polymorphism; and/or results of genotyping for an Hsp60 polymorphism; and/or results of genotyping for a IL-23R polymorphism; and/or results of genotyping for a IL-6 polymorphism; and/or results of genotyping for a IL-17F polymorphism; and interpretation, i.e. prediction. The assessment portion of the report can optionally also include a Recommendation(s). For example, where the results indicate an increased susceptibility to developing CFS, the recommendation can include a recommendation that diet or lifestyle be altered or medical intervention be provided as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., susceptibility prediction).

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of identifying the presence of the target polymorphisms, where such reagents may include nucleic acid primers, arrays of nucleic acid probes, antibodies to polymorphic polypeptides (e.g., immobilized on a substrate), signal producing system reagents, etc., depending on the particular detection protocol to be performed. For example, reagents may include PCR primers that are specific for one or more of the genes TLR4, Hsp60, IL-23R, IL-6 and IL-17F or one or more polymorphisms thereof, as described above. Other examples of reagents include arrays that comprise probes that are specific for one or more of the genes of interest or one or more polymorphisms thereof, and antibodies to epitopes of the proteins encoded by these genes of interest wherein the epitope comprises a polymorphism of interest.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

### EXAMPLE 1

### Subjects and Methods

89 CFS patients (40±12 year old) were enrolled in the study. All were diagnosed for CFS according to the clinical criteria of Fukuda et al. ((1994) Ann. Intern. Med. 121:953-959). 56 aged-matched controls (37±12 year old) were also recruited. 79% of the patients, and 70% of the controls, were females. All subjects were of European origin.

Genomic DNA was extracted from whole blood using the Blood and Tissue DNA extraction kit, Qiagen (Venlo, Netherlands). Analysis of the rs763780 polymorphism in the IL-17F gene was performed with the SNPlex™ genotyping technology, Applied Biosystems (Foster City, Calif., USA). Genotyping analyses were performed by DNAvision S. A. (Charleroi, Belgium), using ISO17025-accredited procedures.

### Results

A significantly lower prevalence of the His161Arg variant was observed in the CFS population as compared to the control population. 8 out of 89 patients have a CT genotype (8.9%), versus 14 out of 56 controls (25%). None of the CFS patients have the CC genotype, versus 2 out of 56 controls (3.6%). In total, 28.5% of the controls have a CC or CT genotype versus only 8.9% of the patients (OR=4.05, p=0.0018).

The observed lower frequency suggests an involvement of IL-17F, and more generally of Th17 cells, in the pathophysiology of CFS. The His161Arg variant antagonizes the pro-inflammatory effects of wild-type IL-17F, and thereby exerts a protective effect against asthma (Kawaguchi et al. (2006) J. Allergy Clin. Immunol. 117(4):795-801). Accordingly, the development and/or maintenance of CFS involves an increased production of IL-17F, and that expression of inactive variants such as His161Arg confer protection against the disease.

Increased IL-17F production has been observed in various inflammatory situations (Kolls and Linden (2004) Immunity 21(4):467-476; Kawaguchi et al. (2006) J. Allergy Clin. Immunol. 117(4):795-801). Interestingly, it also occurs in the context of intestinal disease, such as Crohn's disease or ulcerative colitis (Seiderer et al. (2008) Inflamm. Bowel Dis. 14(4):437-445). Intestinal dysfunction is a common symptom in CFS. Alterations of the intestinal microbial flora have been reported (Logan et al. (2003) Med. Hypotheses 60(6) 915-923); such alterations can lead to intestinal mucosal dysfunction, increased intestinal permeability (leaky gut), etc. that will cause an immune response to the LPS of gram-negative enterobacteria (Maes et al. (2007) J. Affect. Disord. 99(1-3):237-240; Maes et al. (2007) Neuro. Endocrinol. Lett. 28(6):739-44). Exposure of immune cells to LPS could therefore be the clue to the chronic immune activation observed in CFS patients. Considering this hypothesis, it is particularly noteworthy that in addition to IL-6 and TGB-β, optimal Th17 cell induction requires the action of Toll-like receptor (TLR)-activated peripheral blood mononuclear cells (PBMCs). TLR-activated monocytes may contribute to Th17 cell induction by cell-cell contact, through ligation of the T-cell receptor (Evans et al. (2007) Proc. Natl. Acad. Sci. USA 104(43):17034-9); alternatively, TLR-activated PBMCs may secrete a specific set of cytokines that will potentiate Th17 induction (Kattah et al. (2008) Arthritis Rheum. 58(6):1619-29). In this last report, it was shown that TLR-4 (receptor for LPS), as well as TLR-7/8, evoked the most robust induction of Th17, whereas stimulation of TLR-1, -2, -3 or -9 was not efficient.

Th17 cell induction could therefore be the link between exposure to enterobacterial LPS and the symptoms of chronic inflammation associated with CFS. Interestingly, Th17 and Th1 responses are mutually exclusive, since IFN-γ suppresses IL-17 and vice-versa (Kolls and Linden (2004) Immunity 21(4):467-476). Th17 induction is therefore consistent with a decreased production of Th1 cytokines, as seen in CFS. The pro-inflammatory effects of Th17-secreted cytokines are also consistent with other specific dysfunctions observed in CFS patients: IL-17 and IL-22 can disrupt the blood-brain barrier; Th17 lymphocytes transmigrate across the blood-brain barrier endothelial cells and promote inflammation of the central nervous system (Kebir et al. (2007) Nat. Med. 13(10):1173-1175). Blood-brain barrier permeability and CNS inflammation is thought to be a key aspect in the pathogenesis of CFS (Bested et al. (2001) Med. Hypotheses 57(2):231-237).

The implication of Th17 cell activation in the pathogenesis of CFS would be a significant progress in the understanding of the disease, opening new therapeutic perspectives. Other IL-17F-related genes, such as IL-23R, may also play a role in the pathogenesis. IL-17F is indeed specifically produced by IL-23R-expressing Th17 cells; IL-23R is also polymorphic and some variants of this receptor predispose to inflammatory bowel disease (Maloy (2008) J. Intern. Med. 263(6):584-590; Seiderer et al. (2008) Inflamm. Bowel Dis. 14(4):437-445). An association between CFS and polymorphisms of the IL-23R gene, or other functionally related genes, would provide additional support for the implication of the IL-17 axis in the pathogenesis of CFS.

### EXAMPLE 2

### Subjects and Methods

### A. Study Subjects

Study subjects were 107 patients, 40±12 year old, previously diagnosed for CFS according to the clinical criteria of Fukuda et al. (Fukuda et al. 1994; Ann Intern Med 121:953-959), and 64 healthy controls (non-CFS subjects), 37±12 year old. 79% of patients, and 70% of controls, were females. All subjects were of European origin. Patients and controls were selected from medical practices in Oslo, Norway and Brussels, Belgium.

### B. Blood Sampling and Genotyping Procedure

2 ml whole blood was collected in EDTA tubes, and stored at −80° C. until analysis. Genomic DNA was extracted from 200 μl whole blood using the Blood and Tissue DNA extraction kit, Qiagen (Venlo, Netherlands). Analysis of the rs4986790, rs4986791, rs12344353, rs11209026, rs1800795 and rs2340690 polymorphisms was performed with the SNPlex™ genotyping technology, or with Taqman® SNP genotyping assays, both from Applied Biosystems (Foster City, Calif., USA).

### Results

A summary of all genotyping results is provided in Table 1.

TABLE 1
Frequency of specific alleles in controls and in CFS patients

| | | | Hsp60 | | |
|---|---|---|---|---|---|
| Gene | TLR4 | IL-23R | (HSPD1) | IL6 | IL6 + Hsp60 |
| SNP | rs4986790 | rs11209026 | rs2340690 | rs1800795 | Rs1800795 in |
| | rs12344353 | | | | combination |
| | | | | | with rs2340690 |
| Allele | G allele of | A allele | T/T genotype | G/G | T/T genotype of |
| | rs4986790 | | | genotype | rs2340690 in |
| | C allele of | | | | combination |
| | rs12344353 | | | | with G/G |
| | | | | | genotype of |
| | | | | | rs1800795 |
| Frequency | 9% | 0% | 54% | 13% | 4% |
| in controls | | | | | |
| Frequency | 13% | 10% | 74% | 29% | 32% |
| in patients | | | | | |

### A. Toll-Like Receptor 4

Three different SNPs have been analyzed for this gene: rs4986790 (Asp299Gly), rs4986791 (Thr399lle) and rs12344353.

100% cosegregation level for the rs4986790 and rs12344353 SNPs was observed. rs4986791 cosegregrates with the two others, but less systematically (80% only). These high cosegregation levels were already described in the literature.

The G allele of rs4986790 is associated with a lower responsiveness to LPS stimulation. This is detrimental in that affected carriers are more susceptible to gram-negative infections (sepsis); on the other hand, these people tend to develop less inflammation, and seem to have less chance to develop cardiovascular disease or even Alzheimer's disease. TLR4 polymorphisms have been associated with intestinal diseases (Crohn's disease, inflammatory bowel disease), which may be explained by deficient host-bacteria interactions in the gut (Zacharowski et al. (2006) PNAS 103(16):6392-7; Franchimont et al. (2004) Gut 53(7):987-92).

The variant alleles were present in 6/64 (9%) of the controls, versus 14/107 (13%) of the patients (odds ratio 0.5). CFS patients therefore present a higher frequency of the TLR4 variant haplotype.

### B. Heat-Shock Protein 60

Hsp60 is produced in stress situations (oxidative stress, toxic insult); it can be released by the cells and work extra-cellularly as a pro-inflammatory molecule (it is recognized by TLR4 and leads to the proinflammatory activation of macrophages) (Visser et al. (1998) J. Infect. Dis. 177(2):451-454). An intronic C/T substitution (rs2340690) has been described.

C/C or C/T genotypes of rs2340690 were observed in 26% of the patients versus in 46% of the controls (odds ratio 2.3). Therefore, carrying the T/T genotype of rs2340690 is a predisposing factor for CFS.

### C. IL-23R

A SNP located on the IL-23R gene, rs11209026, has already been associated with Crohn's disease (Roberts et al. (2007) Am. J. Gastroenterol 102(12):2754-61). The A allele of this polymorphism was found in 10% of CFS patients, but in none of the controls.

D. IL-6, and IL-6 in Combination with Hsp60

IL-6 polymorphism rs1800795 is another SNP of interest. A higher prevalence of the G/G genotype of rs1800795 was observed in patients than in controls (29% vs. 13%, OR=0.35), indicating that IL-6 polymorphisms are predictive of CFS independent of other polymorphisms, e.g. polymorphisms in other cytokines, e.g. IFNgamma. The G/G genotype of rs1800795 is thought to confer susceptibility to inflammation (Tischendorf et al. (2007) Int J. Immunogenet 34(6):413-8).

A very discriminant genotype is formed by the combination of IL-6 and Hsp60: the frequency of subjects simultaneously carrying the G/G genotype of rs1800795 (IL-6) and the T/T genotype of rs2340690 (Hsp60) was found to be 32% in the patient population, versus only 4% in the control population.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of evaluating a subject for Chronic Fatigue Syndrome (CFS), said method comprising:
(a) genotyping said subject for at least a polymorphism in at least one gene selected from the group consisting of TLR4, Hsp60, IL-23R, IL-6 and IL-17F to obtain a result; and
(b) employing said result to provide an evaluation of a subject for CFS,

2. The method according to claim 1, wherein said TLR4 polymorphism is selected from rs4986790 and rs12344353 and combinations thereof.

3. The method according to claim 2, wherein identification of a G allele of rs4986790 or a C allele of rs12344353 is predictive of susceptibility to CFS or diagnostic of having CFS.

4. The method according to claim 1, wherein said Hsp60 polymorphism is rs2340690.

5. The method according to claim 4, wherein identification of two T alleles of rs2340690 is predictive of susceptibility to CFS or diagnostic of having CFS.

6. The method according to claim 1, wherein said IL-23R polymorphism is rs11209026.

7. The method according to claim 6, wherein identification of an A allele of rs11209026 is predictive of susceptibility to CFS or diagnostic of having CFS.

8. The method according to claim 1, wherein said IL-6 polymorphism is rs1800795.

9. The method according to claim 8, wherein identification of two G alleles of rs1800795 is predictive of susceptibility to CFS or diagnostic of having CFS.

10. The method according to claim 1, wherein said polymorphisms are rs2340890 in Hsp60 and rs1800795 in IL-6.

11. The method according to claim 10, wherein identification of two T alleles of rs2340890 and two G alleles of rs1800795 is predictive of susceptibility to CFS or diagnostic of having CFS.

12. The method according to claim 1, wherein said IL-17F polymorphism is rs763780.

13. The method according to claim 12, wherein identification of a C allele of rs763780 is predictive of resistance to CFS or diagnostic of not having CFS.

14. The method according to claim 1, wherein said evaluation is a prediction of whether a subject is susceptible to Chronic Fatigue Syndrome.

15. The method according to claim 1, wherein said evaluation is a diagnosis of CFS in a a subject suspected of having CFS.

16. The method according to claim 1, wherein said evaluation is a determination of a treatment for a subject with CFS.

17. An assay device for use in determining the CFS susceptibility phenotype of a source of a nucleic acid sample, said kit comprising:
an element for genotyping said sample to identify a polymorphism in at least one gene selected from the group comprising TLR4, Hsp60, IL-23R, IL-6 and IL-17F.

18. The assay device according to claim 17, wherein said TLR4 polymorphism is selected from rs4986790 and rs12344353 and combinations thereof.

19. The assay device according to claim 17, wherein said Hsp60 polymorphism is rs2340690.

20. The assay device according to claim 17, wherein said IL-23R polymorphism is rs11209026.

21. The assay device according to claim 17, wherein said IL-6 polymorphism is rs1800795.

22. The assay device according to claim 17, wherein said IL-17F polymorphism is rs763780.


SEQUENCE LISTING (12 sequences)

SEQ ID NO: 1 - DNA, 5667 bp, homo sapiens
ctcttgctgtttctttagccactggtctgcaggcgttttcttcttctaacttcctctcctgtgacaaaagagataactattagagaaacaaaagtccagaatgctaaggttgccgctttcacttcctctcaccctttagcccagaactgctttgaatacaccaattgctgtggggcggctcgaggaagagaagacaccagtgcctcagaaactgctcggtcagacggtgatagcgagccacgcattcacagggccactgctgctcacagaagcagtgaggatgatgccaggatgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctgaagaggaaaaataaaaacctcctgaggcatttcttgcccagctgggtccaacacttgttcagttaataagtattaaatgctgccacatgtcaggccttatgctaagggtgagtaattccatggtgcactagatatgcagggctgctaatctcaaggagcttccagtgcagagggaataaatgctagactaaaatacagagtcttccaggtgggcatttcaaccaactcagtcaaggaacccatgacaaagaaagtcatttcaactcttacctcatcaagttgaataaagacagagaaaacagaaagagacattgttcttttcctgagtcttttgaatggaaattgtattatgttatagccatcataaaaccattttggtagttttgactgaactgggtgttcactttttcctttttgattgaatacaatttaaattctacttgatgactgcagtcgtcaaggggctcctgatgcaagatgccccttccattttaagtctgtctccttacagaggttaaagtctagtggctaattcctaaggaaacctgattaacacatgctcacaaccatcctggtcattctcgagcatgttctattttttaactaatcacccctgatatatttttatttttatatatccagttttcatttttttacgtcttgcctataagctaatatcataaataaggttgtttaagacgtgcttcaaatatccatattaaccactatttttcaaggaagtatggaaaagtacactctgtcactttgtcactcgatgtcattccaaagttattgcctactaagtaatgactgtcatgaaagcagcattgaaataatttgtttaaagggggcactcttttaaacgggaagaaaatttccgcttcctggtcttatcatggacaatttgggctagaggcaggaaggaagtgggatgacctcaggaggtcaccttttcttgattccagaaacatatgggctgataaacccggggtgacctcatgaaatgagttgcagcagaagtttatttttttcagaacaagtgatgtttgatggacctctgaatctctttagggagacacagatggctgggatccctcccctgtacccttctcactgccaggagaactacgtgtgaaggtattcaaggcagggagtatacattgctgtttcctgttgggcaatgctccttgaccacattttgggaagagtggatgttatcattgagaaaacaatgtgtctggaattaatggggttcttataaagaaggttcccagaaaagaatgttcatccagcctcctcagaaacagaacattcaagaaaaggacaatcaggatgtcatcagggaaatgaaaataaaaaccacaatgagatatcaccttataccaggtagaatggctactataaaaaaatgaagtgtcatcaaggatatagagaaattggaacccttcttcactgctggagggaatggaaaatggtgtagccgttatgaaaaacagtacggaggtttctcaaaaattaaaaatagaactgctatatgatccagcaatctcacttctgtatatatacccaaaataattgaaatcagaatttcaagaaaatatttacactcccatgttcattgtggcactcttcacaatcactgtttccaaagttatggaaacaacccaaatttccattgaaaaataaatggacaaagaaaatgtgcatatacgtacaatgggatattattcagcctaaaaaaagggggaatcctgttatttatgacaacatgaataaacccggaggccattatgctatgtaaaatgagcaagtaacagaaagacaaatactgcctgatttcatttatatgaggttctaaaatagtcaaactcatagaagcagagaatagaacagtggttcctagggaaaaggaggaagggagaaatgaggaaatagggagttgtctaattggtataaaattatagtatgcaagatgaattagctctaaagatcagctgtatagcagagttcgtataatgaacaatactgtattatgcacttaacattttgttaagagggtacctctcatgttaagtgttcttaccatatacatatacacaaggaagcttttggaggtgatggatatatttattaccttgattgtggtgatggtttgacaggtatgtgactatgtctaaactcatcaaattgtatacattaaatatatgcagttttataatatcaattatgtctgaatgaagctataaaaaagaaaagacaacaaaattcagttgtcaaaactggaaatatgaccacagtcagaagtgtttgttactgagtgtttcagagtgtgtttggtttgagcaggtctagggtgattgaacatccctgggtgtgtttccatgtctcatgtactagtgaaagtagatgtgtgcatttgtgcacatatccctatgtatccctatcagggctgtgtgtatttgaaagtgtgtgtgtccgcatgatcatatctgtatagaagagagtgtgattatatttcttgaagaatacatccatttgaaatggatgtctatggctgtttgagatgagttctctactcttgtgcttgtacagtagtctccccttatcccttatgcttggtggatacgttcttagaccccaagtggatctctgagaccgcagatggtaccaaacctcatatatgcaatattttttcctatacataaatacctaagataaagttcatcttctgaattaggcacagtaagagattaacaataactaacaataaaattgaatagttataataatatattgtaataaaagttatgtgaatgtgatctctttctttctctctctcaaaaaaaaaaaaaaaaaa

SEQ ID NO: 2 - protein, 839 aa, homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 3 - DNA, 2339 bp, homo sapiens
acgcggtgccgcggggcgggagtagaggcggagggaggggacacgggctcattgcggtgtgcgccctgcactctgtccctcactcgccgccgacgacctgtctcgccgagcgcacgccttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaaaaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagttaaaaaaaaaaaaaaaaaaaa

SEQ ID NO: 4 - protein, 573 aa, homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 5 - DNA, 2319 bp, homo sapiens
gccccgacgcgcaccgcgattcgccccaagggccctgcgcaggacgctgacgcgaagactcggaggcggaagaaaaaaggagctgtttctaggcttttctaggcgcccagccgagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaaaaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagttaaaaaaaaaaaaaaaaaaaa

SEQ ID NO: 6 - protein, 573 aa, homo sapiens; identical to SEQ ID NO: 4

SEQ ID NO: 7 - DNA, 2826 bp, homo sapiens
acaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 8 - protein, 629 aa, homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 9 - DNA, 1201 bp, homo sapiens
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 10 - protein, 212 aa, homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - DNA, 808 bp, homo sapiens
gaacacaggcatacacaggaagatacattcacagaaagagcttcctgcacaaagtaagccaccagcgcaacatgacagtgaagaccctgcatggcccagccatggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaagaggtgcatatccactcagctgaagaagctgtagaaatgccactccttacccagtgctctgcaacaagtcctgtctgacccccaattccctccacttcacaggactcttaataagacctgcacggatggaaacagaaaatattcacaatgtatgtgtgtatgtactacactttatatttgatatctaaaatgttaggagaaaaattaatatattcagtgctaatataataaagtattaataattt

SEQ ID NO: 12 - protein, 163 aa, homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ